(19) United States
(12) Patent Application Publication
(10) Pub. No.: US 2025/0375312 A1
(43) Pub. Date: Dec. 11, 2025

(54) Natural Mouth Guard

(71) Applicants: Cheryl Smith, North Hollywood, CA (US); Mote London, North Hollywood, CA (US)

(72) Inventors: Cheryl Smith, North Hollywood, CA (US); Mote London, North Hollywood, CA (US)

(21) Appl. No.: 19/203,464

(22) Filed: May 9, 2025

Related U.S. Application Data
(63) Continuation-in-part of application No. 18/361,256, filed on Jul. 28, 2023.

(51) Int. Cl. A61F 5/56; A61L 31/00

(52) U.S. Cl. CPC A61F 5/566; A61L 31/005; A61F 2005/563

(57) ABSTRACT

This invention provides biocompatible alternatives to conventional mouth guards by using natural, non-synthetic materials safe for intraoral contact and ingestion. These materials conform naturally to a wearer's oral anatomy through passive adaptation, integrated features, heat-, pressure-, and/or water-based methods, or manual techniques performed during or after manufacturing, provided all methods and materials remain natural, non-synthetic, and compatible with intraoral use. The materials may exhibit inherent antimicrobial properties or be enhanced through treatment with non-synthetic substances. In one embodiment, long bast plant fibers such as hemp are formed into two dense strips positioned along the sides of the mouth and joined by a flexible, non-synthetic connector permitting angular and width adaptability. In other embodiments, the mouth guard is formed as a unitary configuration composed entirely of natural, non-synthetic materials. The fully natural construction provides a safe, reusable, and anatomically adaptable solution for intraoral protection against grinding, clenching, or impact.

This application is a continuation-in-part of U.S. application Ser. No. 18/361,256, filed Jul. 28, 2023.

### FIELD OF THE INVENTION

This invention relates generally to mouth guards designed to protect the teeth, gums, or oral tissues from damage caused by grinding, clenching, or physical impact, and more particularly to mouth guards made from natural, non-synthetic materials.

### BACKGROUND

Existing mouth guards are typically made from synthetic plastics, such as silicone or thermoplastic, which through grinding and prolonged contact with intraoral surfaces transfer micro-plastics and other toxic substances into the body. Synthetic materials like these have been linked in studies to potential health risks.

This invention solves this by providing a biocompatible alternative comprised of materials safe to ingest and have in contact with intraoral surfaces. It has specific, practical applications in managing teeth grinding, clenching, and oral physical impact without reliance on synthetic plastics or thermoplastics. Its real-world utility lies in providing a reusable and biocompatible alternative to conventional mouth guards, especially for users seeking natural and sustainable options.

### SUMMARY OF THE INVENTION

The invention provides a mouth guard made entirely of natural, non-synthetic materials, as further defined in the Detailed Description below, offering protection for the oral anatomy without exposing a wearer to synthetic plastics or potentially harmful substances commonly found in conventional mouth guards. While the present embodiments are primarily directed toward general oral protection, the invention is not limited to nighttime or dental use and may be adapted for use in contact sports, medical conditions, or other activities requiring intraoral protection.

In one embodiment, the mouth guard comprises two dense strips formed from natural, non-synthetic fibers, positioned to correspond to the left and right sides of a wearer's mouth. Each strip's perimeter may be customized by a wearer—such as by trimming with scissors, tightening a pull-string, or other manual or natural, non-synthetic means—to better fit the anatomical shape of their mouth. The pliability of the natural fibers enables the strips to conform passively to the grooves of a wearer's teeth through repeated wear. A non-rigid, flexible connector made of similar natural, non-synthetic materials joins the two strips, allowing for angular and width adaptability without reliance on synthetic components.

In another embodiment, the mouth guard may be formed as a unitary body made from natural, non-synthetic materials configured to contact the teeth, gums, or inner cheeks; the invention more broadly encompasses all modular or unitary forms—including U-shapes or other geometries—that conform passively to a wearer's mouth or may be customized through integrated features, manual techniques, or the application of heat, water, and/or pressure, provided all methods and materials remain natural, non-synthetic, and compatible with intraoral use.

### BRIEF DESCRIPTION OF THE DRAWINGS

Photographs are submitted as some of the figures in this application. The subject matter—such as the woven structure, fiber composition, and texture of the natural materials—cannot be adequately illustrated in line drawings. Therefore, photographs are deemed necessary to fully convey the invention under 37 CFR 1.84(b)(1).

FIG. 1—Top view of assembled mouth guard.

FIG. 2—Bottom view showing sewn-on connector and variation in material weave.

FIG. 3—Side profile of natural-fiber strips.

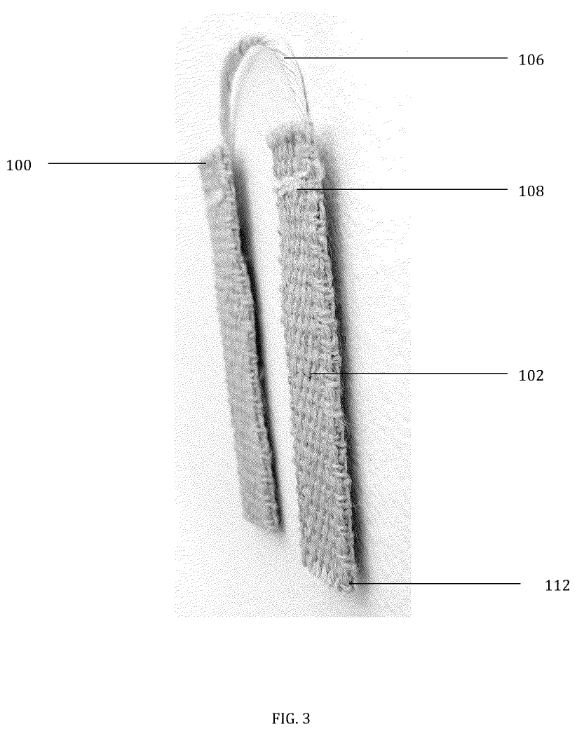

FIG. 4—Handmade prototype showing interlaced structure and woven edge.

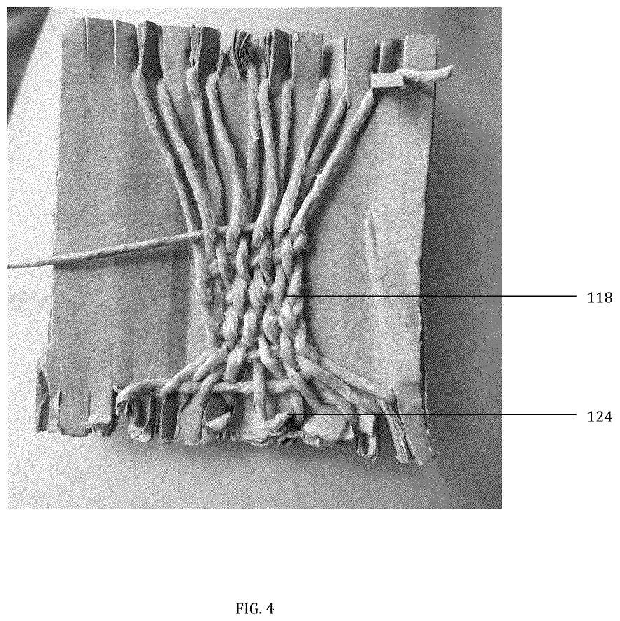

FIG. 5—Completed prototype with woven-in connector.

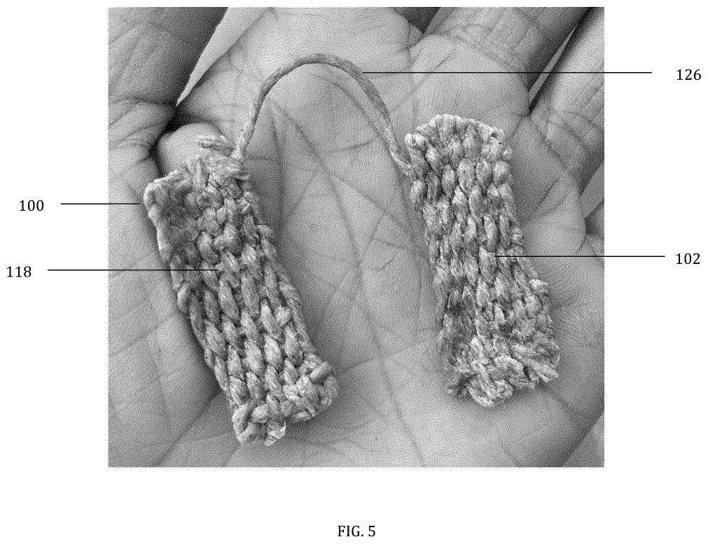

FIG. 6—Worn and trimmed strips with visible bite impressions.

FIG. 7—Double-layer construction with reinforcement stitching.

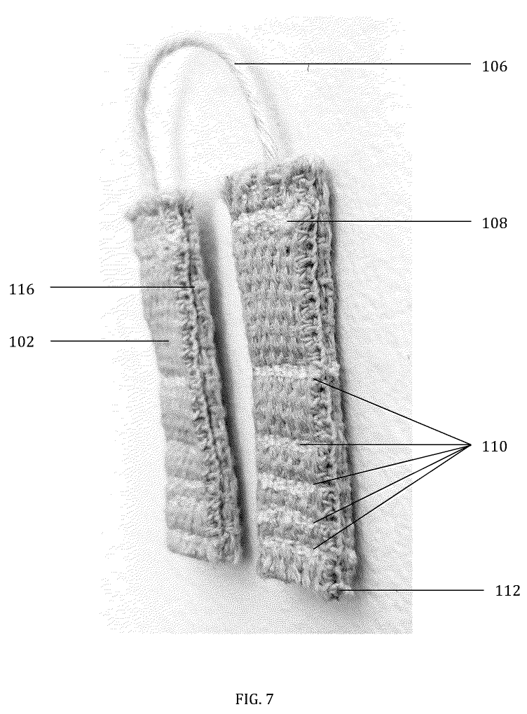

FIG. 8—Trimming guide for wearer customization.

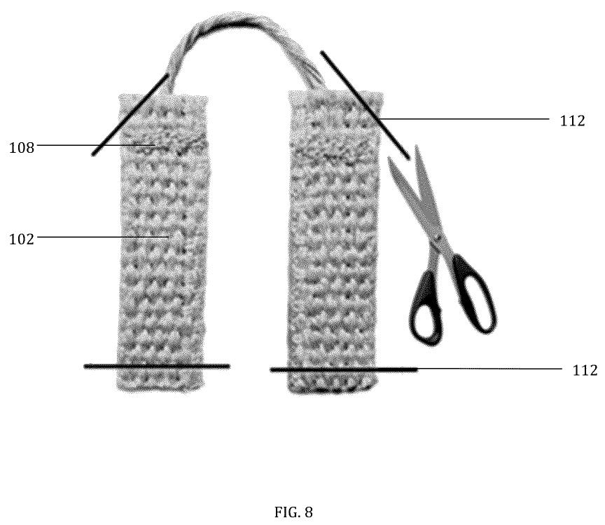

FIG. 9—Mouth guard in use, shown positioned inside a wearer's mouth.

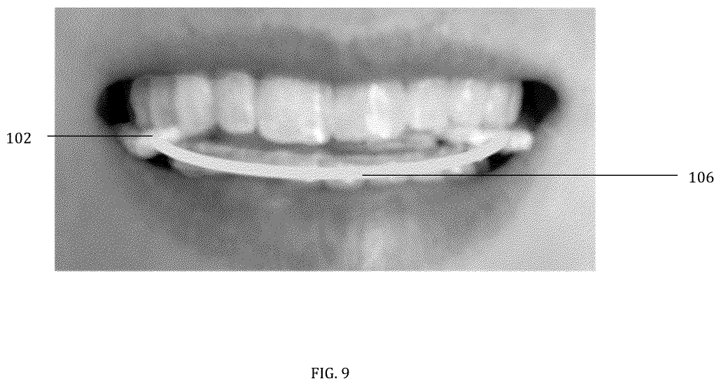

FIG. 10—Product variants demonstrating differences in thickness, softness, fiber density, and woven pattern.

FIG. 11—Reinforcement stitching for trimming and structural durability.

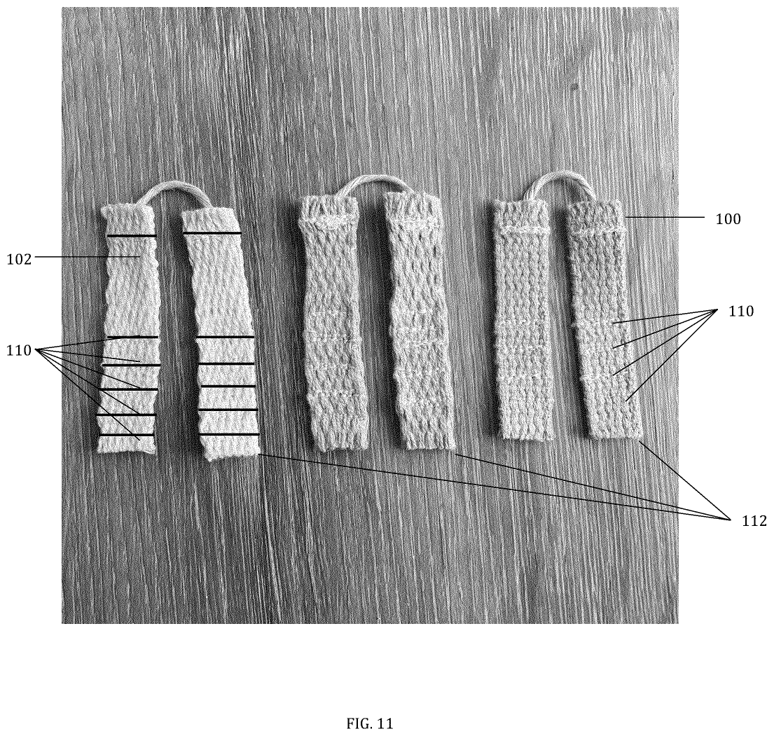

FIG. 12—Trimmed perimeter of the strip, with reinforcement stitching, showing wearer customization.

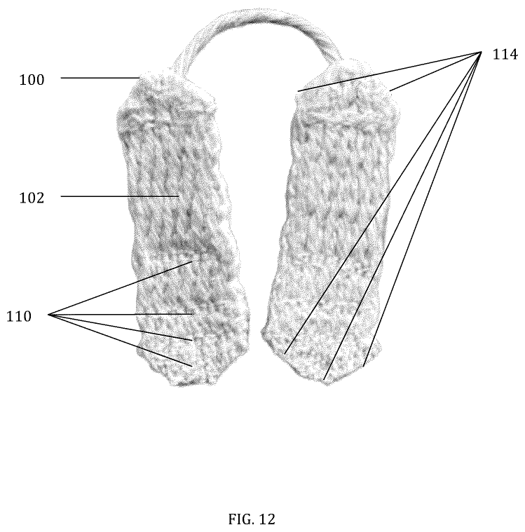

FIG. 13—Angular adaptability of the mouth guard made possible by the flexible connector.

FIG. 14—Width adaptability of the mouth guard enabled by the flexible connector.

FIG. 15—Variations of safety and stability features attached to the connector of a two-strip mouth guard.

FIG. 16—Pull-string variations, showing a two-strip mouth guard before and after tightening, and a unitary configuration with an adjustable perimeter.

### DETAILED DESCRIPTION OF THE INVENTION

The present invention provides a mouth guard (100) constructed entirely of natural, non-synthetic materials. As used herein, “natural, non-synthetic materials” refers to substances derived entirely from natural sources and are free from synthetic polymers, petroleum-based additives, or synthetic chemical processing. This includes plant-based fibers such as hemp, flax, cotton, bamboo, or jute; animal-derived fibers like wool or silk; and sheet-like or structured materials such as bark, cork, palm leaf, or fungal matter. Cotton may be used as a structural material within the invention, provided it is not employed in configurations that function as compressible padding, felt-like laminates, or rigid connector bars. These restricted configurations are addressed in detail below, and are excluded based on functional similarity to prior art—not based on the material itself. Although cotton is generally more moisture-retaining and may degrade more rapidly than other materials, it remains within the scope of the invention when used in structurally appropriate and biocompatible forms.

Unlike traditional guards that rely on synthetic thermoplastics or rigid molded structures, this device conforms to a wearer's mouth through pliable, biocompatible materials that adapt passively over time, through integrated features such as a flexible connectors or flanges, and/or through manual adjustments made by a wearer, such as folding, tying, cinching, looping, tucking, trimming, interlocking, or knotting. In some embodiments, the natural materials may be softened or shaped through the application of heat-, pressure-, and/or water-based methods, provided that the process does not involve synthetic adhesives, compressible padding, or rigid bar formations, and that all shaping methods and materials remain natural, non-synthetic, and compatible with intraoral use. Assembly techniques may also include natural material binding or shaping, such as hand-braiding, wrapping, or pressure-compression using weighted molds, provided no synthetic processing is used. The invention does not rely on compressible padding or fabric-like deformation, but rather on pliable materials that retain structural integrity while conforming through wear or natural shaping techniques.

Suitable natural, non-synthetic materials for the invention include: 1. long bast fibers such as hemp, flax, bamboo, and jute; 2. Seed fibers such as cotton; 3. protein-based animal fibers such as wool or silk; 4. naturally sheet-like or bundled materials such as leather, bark, palm leaf, cork, straw, or fungal substrates like mycelium; 5. naturally derived elastomers such as natural rubber (latex); and 6. emerging biodegradable or bio-based plastics, provided they are free from synthetic polymers, synthetic additives, or petroleum-derived components and are safe for intraoral use. Natural adhesives or binding agents such as tree sap, natural resins, mastic, or chicle may also be used so long as all materials are non-synthetic and biocompatible. Many of these materials also possess inherent antimicrobial properties, which support repeated use and distinguish the invention from disposable and synthetic-based mouth guards. As a peripheral benefit, the invention's reliance on biodegradable materials also contributes to environmental sustainability.

In one preferred embodiment, the mouth guard comprises two dense strips (102) formed from long natural, non-synthetic fibers using a natural technique such as interlacing (e.g., weaving, braiding, knitting, macramé, or the like), wrapping, binding, or other natural methods (118). These strips are positioned to correspond to the left and right sides of a wearer's mouth. The material used for the strips in the present embodiment—commonly referred to as webbing—offers structural durability, fray resistance, and pliability.

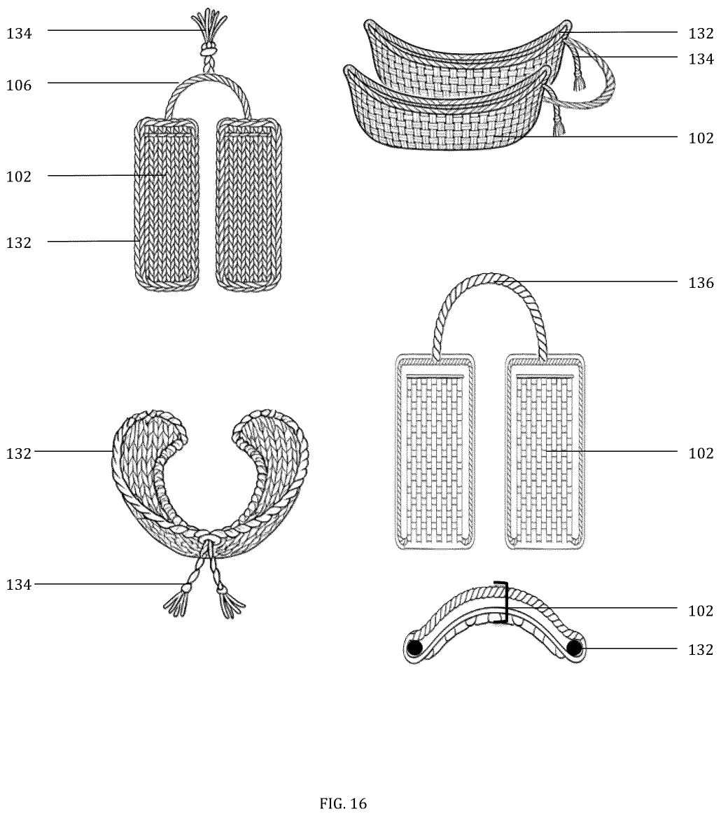

This dense structure is materially distinct from conventional “fabric,” which typically refers to thin, sheet-like textiles as described in prior art mouth guard constructions. In contrast, the dense material used in the present embodiment exhibits increased fiber density, structural thickness, and tensile strength, making it suitable for intraoral use. In addition to providing mechanical resilience, the structure permits wearer customization at the edges. The perimeter of each strip may be trimmed (114) to custom fit a wearer's oral anatomy and maintain a raw edge, or feature a closed pattern (124) that could be customized by cinching the edges with a pull-string (132) as shown in FIG. 16. The pull-string may be adjusted via individual pull-tabs located on each strip (134), via a central pull-tab located on the connector (106, 126), allowing a wearer to simultaneously adjust the connector and the perimeter of the strips, or in some cases the connector is the pull-tab (136).

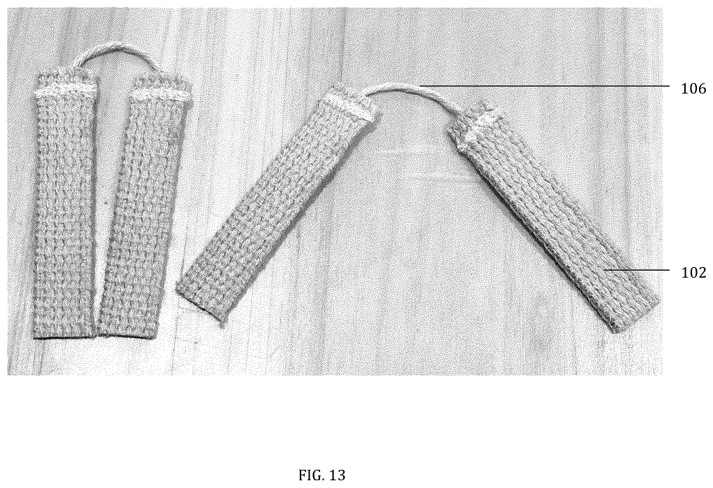

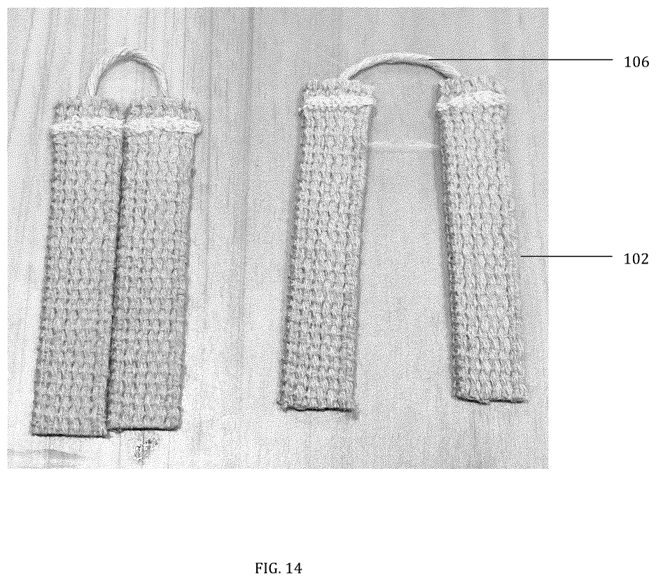

The connector is a flexible, non-rigid linking element that connects the two strips (102) and is composed of natural, non-synthetic materials. This connector may be sewn onto (106) or woven into (126) each strip, or attached using any other method that does not involve synthetic materials or synthetic processes. The connector is not a structural bar or rigid form between the strips as in intraoral appliances in prior art. In contrast, the connector is flexible and configured to permit independent movement and adjustment of the strips relative to each other, which may enable the mouth guard to conform to a wearer's mouth width and angulation (FIGS. 13-14). In some embodiments, the connector is of a fixed length and is configured to rest either in front of or behind a wearer's front teeth depending on their oral anatomy.

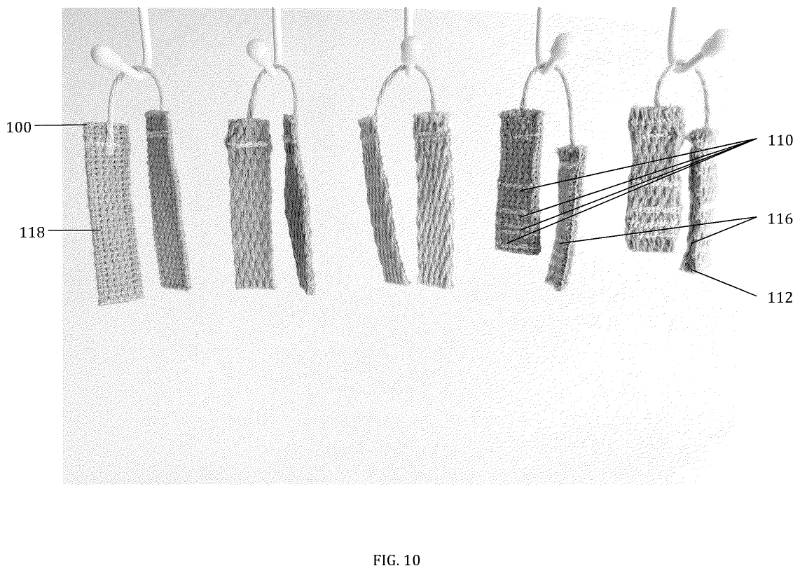

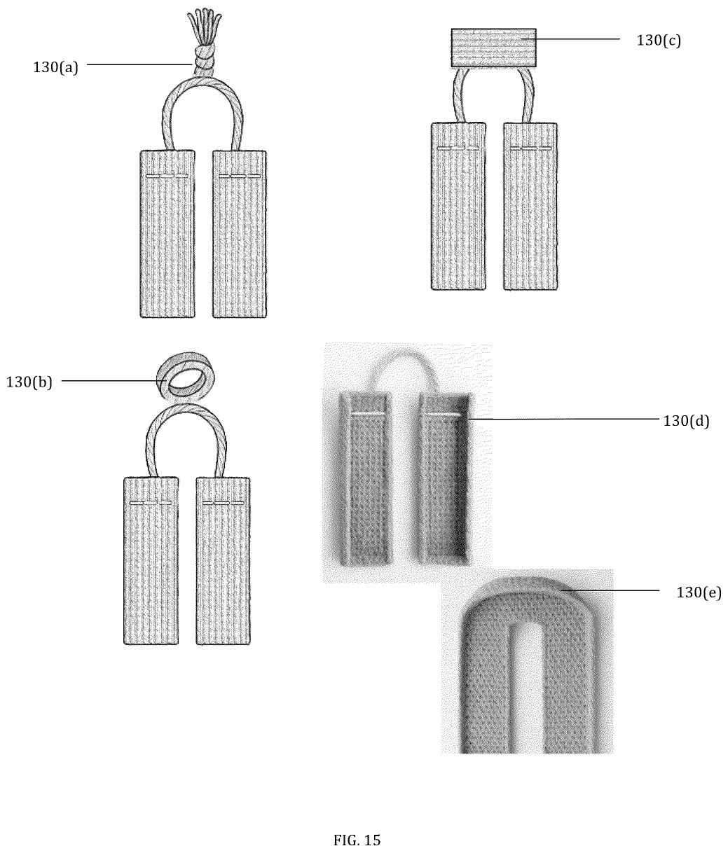

Optional features may include reinforced stitching (110), additional layers (116) for durability, or shallow contours integrated through natural construction techniques. Certain enhancements may also be incorporated, such as tabs, flanges, edges (130d, 130e) or external elements (130a, 130b, 130c) that reduce the risk of dislodgement or choking (FIG. 15). In some embodiments, product variants may differ in thickness, softness, fiber density, or structural configuration while remaining within the scope of the invention, as shown in FIG. 10. In addition to perimeter variations, certain embodiments may include non-peripheral features such as natural ridges, shallow depressions, or internal surface contours that align with intraoral structures or improve comfort, conformity, or retention during use. These features may be naturally formed, trimmed, or integrated during assembly, and remain within the scope of the invention so long as all components consist entirely of natural, non-synthetic materials.

In some embodiments, one or more components of the mouth guard may be coated, infused, or treated with natural, non-synthetic substances to enhance performance, hygiene, or sensory appeal, including natural scent, flavor, or visual distinction. Examples of such substances include beeswax, propolis, plant-based oils, essential oils, chitosan, naturally derived antimicrobials such as silver ions (subject to natural derivation and regulatory compliance), natural flavoring extracts, or natural dyes and pigments (e.g. beet juice, tea leaves, spirulina).

These treatments may improve surface smoothness, reduce microbial colonization, provide an additional barrier to moisture absorption, or enhance taste, freshness, or visual appeal. They may be applied to the connector, strip surfaces, or unitary configuration prior to use, and may be reapplied by a wearer as part of regular maintenance. A wide range of treatments may be employed, provided they consist entirely of natural, non-synthetic substances and do not include synthetic additives or synthetic processes.

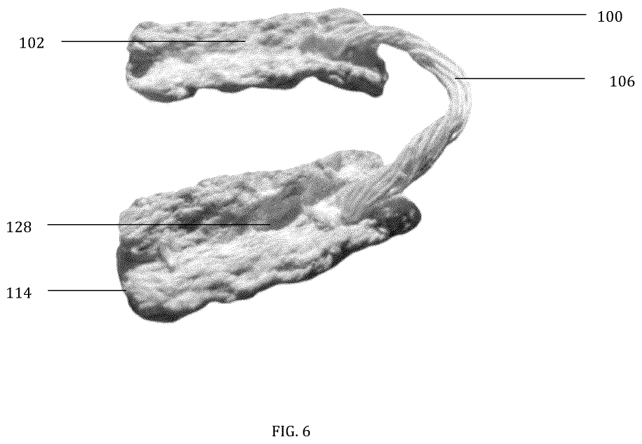

The mouth guard may also be implemented in a unitary configuration or layered assembly of natural, non-synthetic materials, excluding soft fabric laminates or compressible textile padding structures. These configurations may include U-shapes or other geometries designed to protect the teeth, gums, or cheeks. In all forms, the mouth guard is configured to conform to a wearer's anatomy through natural methods such as passive wear, trimming, cinching, pressure, water, heat, or pre-forming, depending on the material type, without relying on synthetic thermoplastics or petroleum-derived compounds. Regardless of configuration, the invention is constructed entirely from natural, non-synthetic materials, and in some embodiments may gradually retain an impression (128) of a wearer's bite over time (FIG. 6).

The mouth guard is not merely a known device made from alternative materials. It introduces an entirely new class of intraoral protection composed exclusively of natural, non-synthetic substances—a configuration not found in prior art and not presently available on the commercial market. Unlike mouth guards constructed from synthetic polymers, soft fabrics, compressible laminates, or rigid plastic or cotton bars, the present invention eliminates all synthetic components while providing structural adaptability, anatomical conformity, and biocompatibility using only materials derived from natural sources.

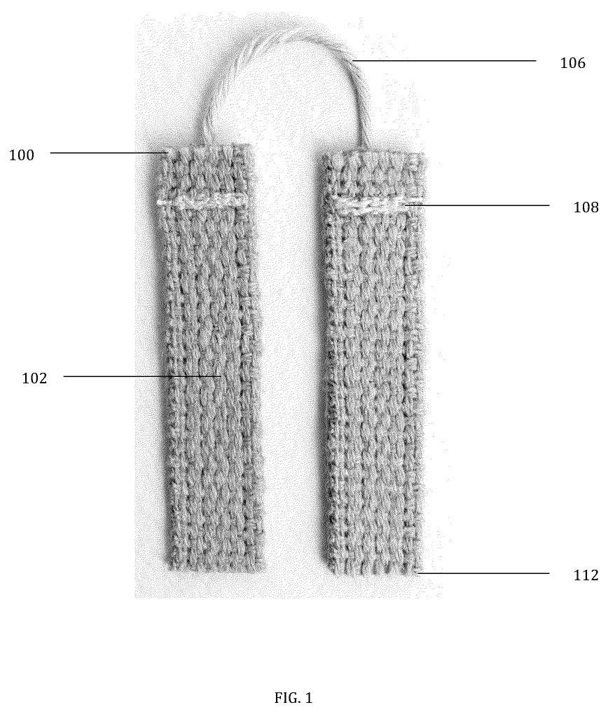

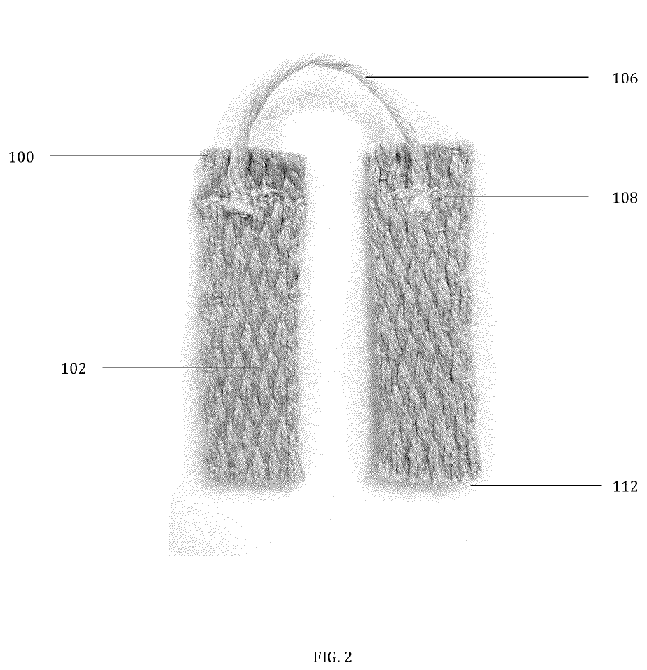

All configurations described herein—including two-strip assemblies (FIGS. 1-2), U-shaped forms in a unitary configuration, layered materials, and pre-contoured variants—fall within the scope of the invention, provided they are made from natural, non-synthetic materials configured for intraoral use.

1. A mouth guard comprising:
(a) two strips made entirely of natural, non-synthetic materials, each strip formed from a structure having sufficient pliability and structural integrity to conform to the left and right sides of a wearer's mouth; and
(b) a flexible connector composed of natural, non-synthetic materials, integrated into each strip by sewing, weaving, or other natural methods that exclude synthetic adhesives or synthetic bonding agents;
wherein the connector is flexible and non-rigid, permitting independent movement of the two strips and, in some embodiments, allowing adaptation to angular or width variations in a wearer's mouth;
and wherein no synthetic polymers, thermoplastics, or synthetic adhesives are used in any component of the mouth guard.

2. A mouth guard comprising:
a modular or unitary structure made entirely of natural, non-synthetic materials;
configured to be worn in the mouth for protection against grinding, clenching, or oral impact;
wherein the structure may include one or more features such as densely interlaced fibers, a trimmable perimeter, a pull-string mechanism, a manually shapeable or customizable form composed of natural, non-synthetic materials, natural antimicrobial or moisture-resistant treatments, a reinforced or woven perimeter edge, integrated channels or openings for connector attachment, or a layered structure composed of natural materials, excluding soft padding or compressible fabric laminates.

3. The mouth guard of claim 1, wherein the natural, non-synthetic materials include, but are not limited to, hemp, flax, cotton, bamboo, jute, wool, silk, leather, bark, cork, palm leaf, straw, fungal substrates, natural rubber (latex), or bio-based plastics, where all such materials are non-synthetic and not subjected to synthetic processing.

4. The mouth guard of claim 2, wherein the natural, non-synthetic materials include, but are not limited to, hemp, flax, cotton, bamboo, jute, wool, silk, leather, bark, cork, palm leaf, straw, fungal substrates, natural rubber (latex), or bio-based plastics, where all such materials are non-synthetic and not subjected to synthetic processing.

5. The mouth guard of claim 1, wherein the natural, non-synthetic materials include one or more bio-based structural substances that exhibit thermoplastic or pliable behavior, allowing the device to be optionally molded by a wearer through the application of heat-, pressure-, and/or water-based methods, provided that all materials remain non-synthetic and free from synthetic polymers, petroleum derivatives, synthetic additives, synthetic adhesives, compressible padding, or rigid bar formations.

6. The mouth guard of claim 2, wherein the natural, non-synthetic materials include one or more bio-based structural substances that exhibit thermoplastic or pliable behavior, allowing the device to be optionally molded by a wearer through the application of heat-, pressure-, and/or water-based methods, provided that all materials remain non-synthetic and free from synthetic polymers, petroleum derivatives, synthetic additives, synthetic adhesives, compressible padding, or rigid bar formations.

7. The mouth guard of claim 1, wherein the structure is sufficiently pliable to gradually conform to a wearer's bite pattern through repeated wear, in the absence of active heat-, pressure-, or water-based shaping.

8. The mouth guard of claim 2, wherein the structure is sufficiently pliable to gradually conform to a wearer's bite pattern through repeated wear, in the absence of active heat-, pressure-, or water-based shaping.

9. The mouth guard of claim 1, wherein the structure includes a perimeter that is manually adjustable by a wearer using non-synthetic techniques, including but not limited to trimming with a scissors or cinching via a built-in pull-string routed around the perimeter edge.

10. The mouth guard of claim 2, wherein the structure includes a perimeter that is manually adjustable by a wearer using non-synthetic techniques, including but not limited to trimming with a scissors or cinching via a built-in pull-string routed around the perimeter edge.

11. The mouth guard of claim 9, wherein the pull-string is configured to draw the perimeter inward, thereby forming a concave shape that improves anatomical fit or retention.

12. The mouth guard of claim 10, wherein the pull-string is configured to draw the perimeter inward, thereby forming a concave shape that improves anatomical fit or retention.

13. The mouth guard of claim 11, wherein the pull-string is routed through both strips and the connector, and may be adjusted via a central pull-tab to simultaneously adjust the connector and cinch the strip edges.

14. The mouth guard of claim 12, wherein the pull-string is routed through both strips and the connector, and may be adjusted via a central pull-tab to simultaneously adjust the connector and cinch the strip edges.

15. The mouth guard of claim 11, wherein the pull-string includes independent pull-tabs on each strip, allowing separate adjustment of the edge geometry without affecting connector length.

16. The mouth guard of claim 12, wherein the pull-string includes independent pull-tabs on each strip, allowing separate adjustment of the edge geometry without affecting connector length.

17. The mouth guard of claim 1, wherein the perimeter comprises a woven or integrated edge structure that supports passive or manual customization, including, but not limited to folding, tying, cinching, looping, tucking, trimming, or knotting.

18. The mouth guard of claim 2, wherein the perimeter comprises a woven or integrated edge structure that supports passive or manual customization, including, but not limited to folding, tying, cinching, looping, tucking, trimming, or knotting.

19. The mouth guard of claim 1, wherein the structure includes raised edges formed by passive adaptation, manual techniques, integrated shaping, or natural, non-synthetic preforming during manufacturing, the raised edges being composed entirely of natural, non-synthetic materials and configured to improve intraoral retention or reduce lateral displacement.

20. The mouth guard of claim 2, wherein the structure includes raised edges formed by passive adaptation, manual techniques, integrated shaping, or natural, non-synthetic preforming during manufacturing, the raised edges being composed entirely of natural, non-synthetic materials and configured to improve intraoral retention or reduce lateral displacement.

21. The mouth guard of claim 1, wherein at least a portion of the mouth guard includes non-peripheral contours, ridges, flexible elements or other surface-adaptive structures configured to align with or support intraoral surfaces during use, and to enhance anatomical conformity, comfort, or retention.

22. The mouth guard of claim 2, wherein at least a portion of the mouth guard includes non-peripheral contours, ridges, flexible elements or other surface-adaptive structures configured to align with or support intraoral surfaces during use, and to enhance anatomical conformity, comfort, or retention.

23. The mouth guard of claim 1, wherein the components are assembled using only natural, non-synthetic materials and natural assembly techniques, including but not limited to stitching, weaving, knotting, folding, wrapping, binding, interlocking, compression fitting, thermal forming of bio-based materials, or the use of natural adhesives such as tree sap, resin, or chicle, wherein the components are free from synthetic plastics, synthetic resins, thermoplastics, or synthetic adhesives.

24. The mouth guard of claim 2, wherein the components are assembled using only natural, non-synthetic materials and natural assembly techniques, including but not limited to stitching, weaving, knotting, folding, wrapping, binding, interlocking, compression fitting, thermal forming of bio-based materials, or the use of natural adhesives such as tree sap, resin, or chicle, wherein the components are free from synthetic plastics, synthetic resins, thermoplastics, or synthetic adhesives.

25. The mouth guard of claim 1, wherein the natural materials used possess inherent antimicrobial, antibacterial, or antifungal, or low moisture retention properties, and are structurally resilient to permit repeated use without physical or dimensional degradation, or are treated with natural substances that enhance such properties while maintaining biocompatibility and structural integrity.

26. The mouth guard of claim 2, wherein the natural materials used possess inherent antimicrobial, antibacterial, or antifungal, or low moisture retention properties, and are structurally resilient to permit repeated use without physical or dimensional degradation, or are treated with natural substances that enhance such properties while maintaining biocompatibility and structural integrity.

27. The mouth guard of claim 1, wherein one or more portions are treated, coated, or infused with natural substances including, but not limited to, beeswax, propolis, plant-based oils, essential oils, natural dyes and pigments, or other non-synthetic substances, to enhance antimicrobial performance, reduce moisture absorption, improve intraoral durability or comfort, impart natural scent, flavor, appearance or other sensory characteristics that increase user acceptability or commercial appeal.

28. The mouth guard of claim 2, wherein one or more portions are treated, coated, or infused with natural substances including, but not limited to, beeswax, propolis, plant-based oils, essential oils, natural dyes and pigments, or other non-synthetic substances, to enhance antimicrobial performance, reduce moisture absorption, improve intraoral durability or comfort, impart natural scent, flavor, appearance or other sensory characteristics that increase user acceptability or commercial appeal.

29. The mouth guard of claim 1, wherein each strip is formed into a dense structure using natural techniques including but not limited to interlacing, weaving, braiding, knitting, wrapping, or binding, and is materially distinct from conventional fabric by exhibiting increased fiber density, structural thickness, and tensile strength.

30. The mouth guard of claim 1, wherein the connector is of fixed or adjustable length and configured to rest in front of or behind a wearer's front teeth depending on anatomical fit.

31. A method of fitting a mouth guard comprising:
providing a mouth guard composed entirely of natural, non-synthetic materials, the mouth guard comprising a modular structure—including but not limited to a two-strip configuration—or a unitary structure shaped to conform to a wearer's oral anatomy;
placing the mouth guard inside a wearer's mouth such that it aligns with the teeth, gums, or inner cheeks;
and customizing the mouth guard to a wearer's anatomy using natural, non-synthetic techniques, including but not limited to trimming, pull-string adjustment, heat-, pressure-, and water-based shaping, and other passive, integrated, or manual modification methods.

32. A method of manufacturing a mouth guard comprising:
constructing a structure entirely from natural, non-synthetic materials, the structure being created using one or more techniques including, but not limited to:
(a) forming long natural fibers into structural components using a natural technique such as interlacing, weaving, knitting, braiding, wrapping, binding, or other natural techniques;
(b) layering or shaping a unitary or modular configuration to correspond to intraoral anatomy;
and assembling the mouth guard using only natural, non-synthetic materials and natural, non-synthetic attachment methods that exclude synthetic adhesives, synthetic bonding agents, or synthetic processing techniques;
wherein the resulting mouth guard is configured to protect the teeth, gums, or oral surfaces and adapt to a wearer's mouth through passive, manual, integrated, or other non-synthetic fitting techniques.

33. The method of claim 32, wherein all materials are non-synthetic and the assembly process excludes synthetic adhesives and petroleum-based substances, thereby enabling small-scale, artisanal, or scalable production using natural, non-synthetic components.
